(19) United States
(12) Patent Application Publication
(10) Pub. No.: US 2022/0287680 A1
(43) Pub. Date: Sep. 15, 2022

(54) Shape Memory Support For Interventional Device Insertion Tubes

(71) Applicant: GE Precision Healthcare LLC, Wauwatosa, WI (US)

(72) Inventors: Birger Løype, Horten (NO); Serge Calisti, Marseille (FR)

(21) Appl. No.: 17/195,965

(22) Filed: Mar. 9, 2021

(51) Int. Cl. A61B 8/00; A61B 8/12

(52) U.S. Cl. CPC A61B 8/4455; A61B 8/12

(57) ABSTRACT

An invasive/interventional device or probe for use in an interventional medical procedure includes a control handle operably connected to an imaging system and an insertion tube. The insertion tube includes a first, proximal section operably connected to the control handle and a second, distal section operably connected to the first section opposite the control handle. The first section includes a stiff but flexible structure that can be manipulated to alter the shape of the first section, but that can retain the selected shape after being moved. The nature of the construction for the first section enables the first section to positioned as desired by the operator of the probe, and to retain that position until modified by the operator. The first section of the insertion tube allows the operator to manipulate only the second section during the procedure, reducing operator fatigue and increasing positional precision for the probe.

### BACKGROUND OF THE INVENTION

Embodiments of the present disclosure relate generally to interventional imaging and, more particularly, to structures of the interventional imaging probes and their method of operation used in these interventional procedures.

Various medical conditions affect internal organs and structures. Efficient diagnosis and treatment of these conditions typically require a physician to directly observe a patient's internal organs and structures. For example, diagnosis of various heart ailments often requires a cardiologist to directly observe affected areas of a patient's heart. Instead of more intrusive surgical techniques, ultrasound imaging is often utilized to directly observe images of a patient's internal organs and structures.

By way of example, interventional procedures such as transesophageal echocardiography (TEE) and/or intracardiac echocardiography (ICE) may be used to provide high resolution images of intracardiac anatomy. The high-resolution images, in turn, allow for real-time guidance of interventional devices during structural heart disease (SHD) interventions such as transcatheter aortic valve implantation (TAVI), paravalvular regurgitation repair, and/or mitral valve interventions.

TEE procedures are typically performed in examination, intervention and operating room (open heart surgery) situations where imaging of internal structures of the patient is required. The device utilized in performing TEE typically includes an invasive or interventional device or probe, a processing unit, and a monitor. The probe is connected to the processing unit which in turn is connected to the monitor. In operation, the processing unit sends a triggering signal to the probe. The probe then emits ultrasonic signals via an imaging element within the probe into the patient's heart. The probe then detects echoes of the previously emitted ultrasonic signals. Then, the probe sends the detected signals to the processing unit which converts the signals into images. The images are then displayed on the monitor. The probe typically includes a semi-flexible insertion tube that includes a transducer located near the end of the probe.

Typically, during TEE, the insertion tube is introduced into the mouth of a patient and positioned in the patient's esophagus. The insertion tube is then positioned so that the transducer is in a position to facilitate heart imaging. That is, the insertion tube is positioned so that the heart or other internal structure to be imaged is in the direction of view of the imaging element or transducer disposed within the insertion tube. Typically, the transducer sends ultrasonic signals through the esophageal wall that come into contact with the heart or other internal structures. The transducer then receives the ultrasonic signals as they bounce back from various points within the internal structures of the patient. The transducer then sends the received signals back through the insertion tube typically via wiring. After the signals travel through the insertion tube and probe, the signals enter the processing unit typically via wires connecting the probe to the processing unit.

Often, in addition to the heart, it may be desirable to image other internal structures within the body of a patient using other interventional imaging procedures and devices, including bronchoscopes or colonoscopes, for example. Imaging other internal structures may require re-positioning or use of a different probe in order to view the internal organs or other internal structures of the patient that are desired. Additionally, viewing the heart and/or other internal structures from various angles and perspectives may require re-positioning of the probe during these procedures.

Although TEE allows for well-defined workflows and good image quality, TEE may not be suitable for all cardiac interventions. Accordingly, in other interventional procedures, ICE may be used to provide high resolution images of cardiac structures, often under conscious sedation of the patient. Furthermore, ICE equipment, which utilizes probes highly similar in construction to those used for TEE, may be interfaced with other interventional imaging systems, thus allowing for supplemental imaging that may provide additional information for device guidance, diagnosis, and/or treatment. For example, a CT, MRI, PET, ultrasound, fluoroscopy, electrophysiology, and/or X-ray imaging system may be used to provide supplemental views of an anatomy of interest in real-time to facilitate ICE-assisted interventional procedures.

In either of these procedures or in any similar invasive or interventional procedure, as previously discussed, the probe or interventional device inserted into the patient includes a control handle with an elongate, flexible insertion tube extending outwardly from the handle. The tube encloses a suitable movement mechanism that is operably connected to a control device on the control handle, such that an operator can control the movement of the mechanism, and the movement of the flexible tube, within the patient. Opposite the control handle, the flexible insertion tube includes an imaging element that is operable to obtain the ultrasound images of the anatomy of the patient.

In order to accommodate the variations in the size of individual patients, the flexible insertion tube has a length that enables the imaging element to be positioned where necessary to provide the ultrasound images of the patient anatomy. Typically, this length for the flexible insertion tube is approximately one (1) meter.

While this length for the flexible insertion tube provides various advantages with regard to the utilization of the invasive device in an interventional procedure, certain drawbacks are also present. More specifically, the length of the flexible insertion tube requires that the movement mechanism and other wires and associated items for the operation of the imaging element extend along the entire length of the flexible tube. With the length of all of these required components within the flexible tube, the weight of the tube is significantly increased. Thus, with the flexible insertion tube being formed to have the desired flexibility for proper insertion and manipulation within the patient, the construction of the flexible insertion tube is unable to reliably hold the imaging element in a stable position due to the weight of the tube and the internal components within the insertion tube

In addition, with the added weight of the control handle, the overall weight of the apparatus that must be supported and manipulated by the operator during an interventional procedure is significant. Further, as the length of time the operator must hold the apparatus can be as long as three (3) hours for certain procedures, the fatigue generated in the operator by holding and utilizing the apparatus for this amount of time is greatly increased.

Therefore, it is desirable to develop a structure for an invasive/interventional device or probe utilized in an interventional medical procedure that can significantly reduce any instability in the positioning of the device when in operation. Further, the improved invasive/interventional device or probe structure should limit or reduce the fatigue of the operator due to the use of the probe in the procedure.

### BRIEF DESCRIPTION OF THE DISCLOSURE

In the present disclosure an invasive/interventional device or probe for use in examination, open heart surgery and interventional medical procedures includes a control handle operably connected to an imaging system and an insertion tube. The insertion tube includes a first, proximal section operably connected to the control handle and a second, distal section operably connected to the first section opposite the control handle. The second section is formed as a flexible tube including a number of internal passages within which various operating components of the probe can extend between the first section and various movement control and imaging structures disposed within the second section. The first section is formed with a structure that can be moved to alter the shape of the first section, but that can retain the selected shape after being moved. The semi-rigid nature of the first section enables the first section to positioned as desired by the operator of the probe, and to retain that position until modified by the operator. In this manner the first section of the insertion tube allows the operator to manipulate only the second section of the insertion tube during the procedure, reducing operator fatigue and increasing imaging precision for the probe.

According to another exemplary aspect of the disclosure, the insertion tube includes a support structure secured thereto. The support structure is disposed around the exterior of the insertion tube adjacent the control handle and engages a proximal section of the insertion tube, with a distal section of the insertion tube extending outwardly of the support structure for contact with the patient. The support structure is formed with a stiff but flexible construction that can be manipulated into various configurations but that can also maintain its shape after being manipulated into the desired shape. The support structure can be releasably disposed around the proximal section of the insertion tube in order to enable the proximal section to be moved into a desired shape or position along with the support structure and maintain that position as a result of the stiffness of the support structure.

In one exemplary embodiment of the invention, an insertion tube assembly for an interventional medical device, the insertion tube assembly including a first section adapted to be secured to a handle of the interventional device and forming a proximal end of the insertion tube, and a second section operably connected to the first section and forming a distal portion of the insertion tube adapted for insertion into an object, wherein the first section has a stiffness greater than the second section.

In another exemplary embodiment of the invention, an interventional medical device includes a control handle, and an insertion tube assembly operably connected to the control handle, wherein the insertion tube assembly has a first section connected to the control handle and forming a proximal end of the insertion tube, and a second section operably connected to the first section opposite the handle and forming a distal portion of the insertion tube adapted for insertion into an object, wherein the first section has a stiffness greater than the second section.

In still another exemplary embodiment of the method of the invention, a method for adjusting the position of an insertion tube of an interventional medical device during use in an interventional procedure for a patient including the steps of providing interventional medical device having a control handle, and an insertion tube assembly operably connected to the control handle, wherein the insertion tube assembly has a first section, and a second section operably connected to the first section, wherein the first section has a stiffness greater than the second section, manipulating the first section to position the first section in a desired configuration, and manipulating the second section to position the second section within the patient at a desired location to obtain images of internal structures of the patient, wherein the first section remains substantially stationary during manipulation of the second section.

It should be understood that the brief description above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

FIG. 1 is a schematic representation of an exemplary imaging system, in accordance with aspects of the present disclosure.

FIG. 2 is an isometric view of an interventional device including an insertion tube according to an exemplary embodiment of the disclosure and usable with the system illustrated in FIG. 1.

FIG. 3 is a schematic view of the insertion tube and the interventional device of FIG. 2 being utilized in an interventional procedure according to an exemplary embodiment of the disclosure.

FIG. 4 is a cross-sectional view of a first section of the insertion tube in accordance with an exemplary embodiment of the disclosure.

FIG. 5 is a schematic view of a first section of the insertion tube in accordance with another exemplary embodiment of the disclosure.

FIG. 6 is a schematic view of an insertion tube including a support structure in accordance with another exemplary embodiment of the disclosure.

### DETAILED DESCRIPTION

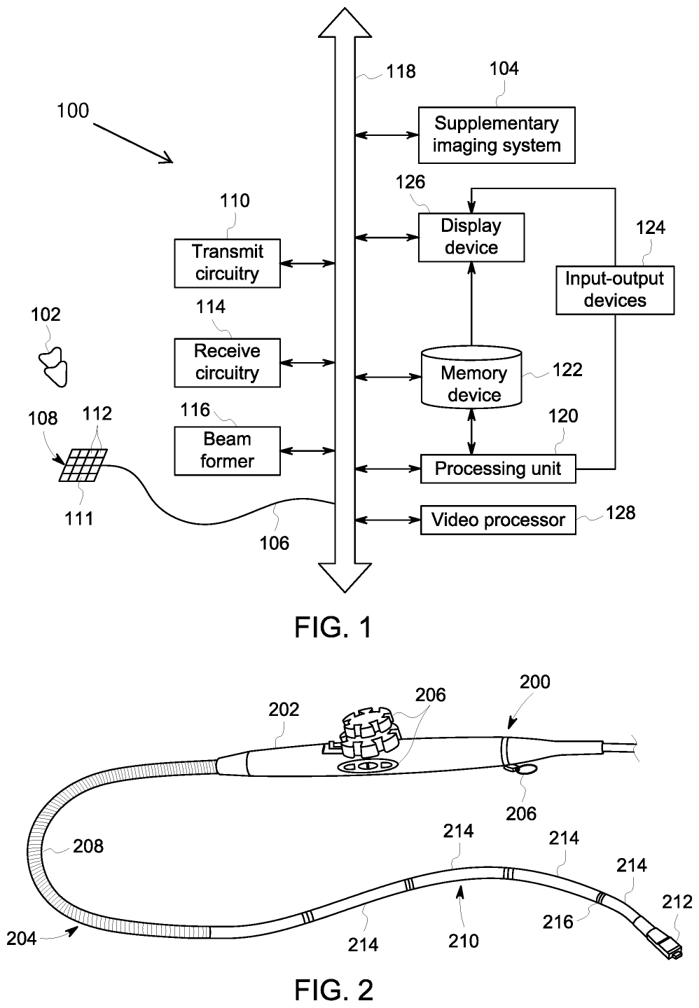

FIG. 1 illustrates an exemplary imaging system 100 for optimal visualization of a target structure 102 for use during interventional procedures. For discussion purposes, the system 100 is described with reference to a TEE system. However, in certain embodiments, the system 100 may be implemented in other interventional imaging systems such as a TTE system, a ICE system, an OCT system, a magnetic resonance imaging (MRI) system, a CT system, a positron emission tomography (PET) system, and/or an X-ray system. Additionally, it may be noted that although the present embodiment is described with reference to imaging a cardiac region corresponding to a patient, certain embodiments of the system 100 may be used with other biological tissues such as lymph vessels, cerebral vessels, and/or in non-biological materials.

In one embodiment, the system 100 employs ultrasound signals to acquire image data corresponding to the target structure 102 in a subject. Moreover, the system 100 may combine the acquired image data corresponding to the target structure 102, for example the cardiac region, with supplementary image data. The supplementary image data, for example, may include previously acquired images and/or real-time intra-operative image data generated by a supplementary imaging system 104 such as a CT, MRI, PET, ultrasound, fluoroscopy, electrophysiology, and/or X-ray system. Specifically, a combination of the acquired image data, and/or supplementary image data may allow for generation of a composite image that provides a greater volume of medical information for use in accurate guidance for an interventional procedure and/or for providing more accurate anatomical measurements.

Accordingly, in one embodiment, the system 100 includes an interventional device or probe 106 such as an ultrasound probe, a laparoscope, a bronchoscope, a colonoscope, a needle, a catheter and/or an endoscope. The interventional device 106 is adapted for use in a confined medical or surgical environment such as a body cavity, orifice, or chamber corresponding to a subject, e.g., a patient. The interventional device 106 may further include at least one imaging subsystem 108 disposed at a distal end of the interventional device 106. The imaging subsystem 108 may be configured to generate cross-sectional images of the target structure 102 for evaluating one or more corresponding characteristics. Particularly, in one embodiment, imaging subsystem 108 is configured to acquire a series of three-dimensional (3D) and/or four-dimensional (4D) ultrasound images corresponding to the subject, though the subsystem 108 can also obtain one-dimensional (1D) and two-dimensional (2D) ultrasound images. In certain embodiments, the system 100 may be configured to generate the 3D model relative to time, thereby generating a 4D model or image corresponding to the target structure such as the heart of the patient. The system 100 may use the 3D and/or 4D image data, for example, to visualize a 4D model of the target structure 102 for providing a medical practitioner with real-time guidance for navigating the probe/interventional device 106 within the patient.

To that end, in certain embodiments, the imaging subsystem 108 can be an ultrasound imaging system that includes transmit circuitry 110 that may be configured to generate a pulsed waveform to operate or drive an imaging element 111, such as one or more transducer elements 112. The transducer elements 112 are configured to transmit and/or receive ultrasound energy and may comprise any material that is adapted to convert a signal into acoustic energy and/or convert acoustic energy into a signal. For example, the transducer elements 112 may be a piezoelectric material, such as lead zirconate titanate (PZT), or a capacitive micromachined ultrasound transducer (CMUT) according to exemplary embodiments. The interventional device 106 may include more than one transducer element 112, such as two or more transducer elements 112 arranged in an array, or separated from each other on the interventional device 106. The transducer elements 112 produce echoes that return to the transducer elements 112 and are received by receive circuitry 114 for further processing. The receive circuitry 114 may be operatively coupled to a beamformer 116 that may be configured to process the received echoes and output corresponding radio frequency (RF) signals.

Further, the system 100 includes a processing unit 120 communicatively coupled to the acquisition subsystem, to operatively connect the processing unit 120 to the beamformer 116, the interventional device 106, and/or the receive circuitry 114, over a wired or wireless communications network 118. The processing unit 120 may be configured to receive and process the acquired image data, for example, the RF signals according to a plurality of selectable ultrasound imaging modes in near real-time and/or offline mode.

Moreover, in one embodiment, the processing unit 120 may be configured to store the acquired volumetric images, the imaging parameters, and/or viewing parameters in a memory device 122. The memory device 122, for example, may include storage devices such as a random access memory, a read only memory, a disc drive, solid-state memory device, and/or a flash memory. Additionally, the processing unit 120 may display the volumetric images and or information derived from the image to a user, such as a cardiologist, for further assessment on a operably connected display 126 for manipulation using one or more connected input-output devices 124 for communicating information and/or receiving commands and inputs from the user, or for processing by a video processor 128 that may be connected and configured to perform one or more functions of the processing unit 120. For example, the video processor 128 may be configured to digitize the received echoes and output a resulting digital video stream on the display device 126.

Referring now to FIG. 2, the interventional device 106 is disclosed in the illustrated exemplary embodiment as being formed as an ultrasound probe/TEE probe 200. The ultrasound probe 200 includes a control handle 202 that is operatively connected to the processing unit, and an insertion tube 204 extending outwardly from the control handle 202. The control handle 202 includes one or more control elements 206 thereon that enable the operator of the ultrasound probe 200 to control the various operations of the internal movement and imaging mechanisms and associated wiring and/or other connections (not shown) disposed within hollow interior of the insertion tube 204.

Looking now at FIGS. 2-4, the insertion tube 204 is formed of a first section 208 and a second section 210. The first section 208 is operably engaged with the control handle 202, and in certain exemplary embodiments is releasably engaged with the control handle 202, and extends from the control handle 202 to form a proximal portion of the insertion tube 204 that is configured to remain outside of the patient 213. The second section 210 is connected to the first section 208 at one end opposite the handle 202 to form a distal portion of the insertion tube 204, and terminates in a tip 212 at the opposite end, in which the imaging element 111 can be disposed. The second section 210 is designed for insertion into the body of a patient 213, such as into the esophagus of the patient 213, and can be formed in any suitable manner to be usable for this purpose, such as with independently articulable segments 214 joined to one another and covered by a fluid impervious sheath 216 suitable for patient contact to facilitate the insertion, movement and positioning of the second section 210 as desired within the patient.

While the second section 210 is formed to be highly flexible in order to facilitate insertion into the patient 213, the first section 208 is formed in a manner to render the first section 208 much stiffer, semi-rigid, or less flexible than the second section 210. By increasing the stiffness of the first section 208 relative to the second section 210, the first section 208 can be constructed to be able to more stably hold the position of the second section 210 when the insertion tube 204 is inserted within the patient 213.

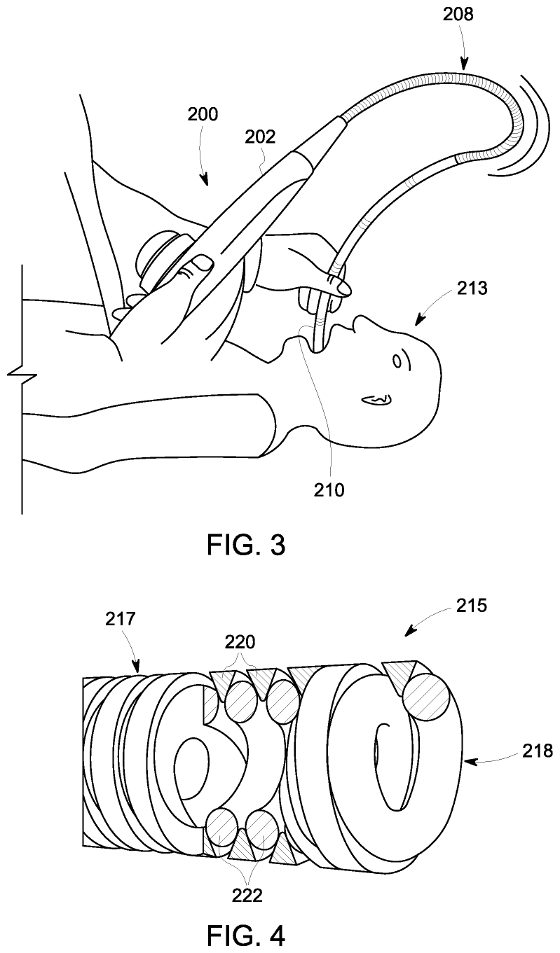

In the illustrated exemplary embodiments of FIGS. 3 and 4, the first section 208 is forms a suitable length of the insertion tube 204 to provide the desired stability to the position of the second section 210. In one exemplary embodiment, the first section 208 can be up to 60 cm in length. In other exemplary embodiments, the first section 208 can have a length of up to 40 cm of the insertion tube 204 extending from the control handle 202. The positioning of the first section 208 additionally provides a gradual transition in the stiffness of the interventional device/ultrasound probe 200 from the rigid control handle 202, through the flexible but stiff first section 208 to the highly flexible second section 210.

In many situations, due to the presence of the semi-rigid first section 208, the overall length of the insertion tube 204 can be increased, creating a longer distance between the operator and the patient 213. The longer insertion tube 204 that can be employed when the semi-rigid first section 208 forms a portion of the tube 204 enables the user to position themselves further from the patient 213, reducing the congestion of individuals in the immediate vicinity of the patient 213 during any procedure being performed on the patient 213. In addition, the increased spacing from the patient 213 provided by the insertion tube 204 having the semi-rigid first portion 208 allows the user to be positioned outside of the range of the components (e.g., a C-arm) or radiation beams emitted by any supplementary imaging system used along with the insertion tube 204 in the procedure.

This can be particularly advantageous when the control handle 202 is disposed within a holder (not shown). The holder is a mechanical structure that supports the control handle 202 near the patient 213 but separate from the user to eliminate inadvertent movement of the insertion tube 204 as a result of the motion of the user holding the control handle 202. In the holder, the control handle 202 is maintained stationary, and the use of the semi-rigid first section 208 further minimizes inadvertent movement of the insertion tube 204 as a result of the semi-rigid nature of the construction 215 of the first section 208 pursuant to this exemplary embodiment, or of any separate component or structure utilized with the first section 208 to provide a semi-rigid aspect to the first section 208 pursuant to any other exemplary embodiment of this disclosure.

To provide the increased stiffness desired, in one exemplary embodiment shown in FIG. 4, the first section 208 has a flexible, shape-retaining construction 215 enabling the first portion 208 to be movably positioned in a number of different configurations while the construction of the first section 208 enables the first section 208 to retain its position in that configuration. Further, the stiffness of the construction 215 of the first section 208 enables the second section 210 of the insertion tube 204 to be manipulated by the user without significantly shifting the configuration of the first section 208. In this manner, the first section 208 can be positioned as desired by the user, and will remain in the desired position to provide support to the second section 210 as the second section 210 is manipulated by the user in the interventional procedure. This support for the second section 210 provided by the first section 208 enhances the ability of the second section 210 and the imaging element 111 to be precisely positioned within the patient by the user and also reduces fatigue to the user by lessening the weight of the insertion tube 204 that must be independently supported by the user. As a result, the construction for the insertion tube 204 with the first section 208 allows for better images to be obtained using the endoscope 200 with less effort as opposed to prior art endoscopes and associated insertion tubes.

The flexible, shape-retaining construction 215 for the first section 208 to provide the desired combination of flexibility and stiffness can have a number of different suitable variations. In one exemplary embodiment illustrated in FIG. 4, the first section 208 has a gooseneck construction 217 with a round spring core 218 and a triangle rod 220 disposed between sections 222 of the core 218, though other numbers of separate cores and/or rods 222 could also be utilized, such as in separate but interconnected gooseneck section 217. The core 218 and rods 220 are formed from any suitable material or materials, which can be the same or different from one another, such as a metal, and in particular a steel, brass or aluminum. The gooseneck construction 217 enables the first section 215 to be manipulated as desired to position the first section 215 in a desired configuration for use of the insertion tube 204. Additionally, due to the nature and shape of the round spring core 218 and the position of the rod 220, the gooseneck construction 217 provides a shape-retaining structure that can provides a stable hollow interior/central passage through the gooseneck construction 217, to prevent crimping of the wiring or other connections running through the first section 215 of the insertion tube 204. The core 218 and rods 220 can additionally be covered by a suitable sheath (not shown) in order the enclose the core 218 and rods 220 to aid in the manipulation, ease of use and cleaning of the first section 208. Further, in one exemplary embodiment the first section 208 and the second section 210 are manufactured as one part to form the insertion tube 204, the metal structures can be connected by welding, brazing, soldering or gluing, among other suitable connections, and subsequently covered or enclosed within a flexible plastic and fluid-impervious material, e.g., a thermoplastic polyurethane. In other exemplary embodiments the first section 208 and second section 210 can be formed separately from one another and subsequently joined or connected to one another in any suitable known manner when employed in a procedure.

With this construction 217 for the first section 208, the first section 208 can be manipulated by the user into to virtually desired straight or bent position, which will be held by the first section 208 when released by the user. The stiffness of the construction 217 of the first section 208 is sufficient to hold the desired configuration during further manipulation of the second section 210 by the user during the procedure, but the position of the first portion 208 can readily be altered by direct manipulation of the first section 208 by the user.

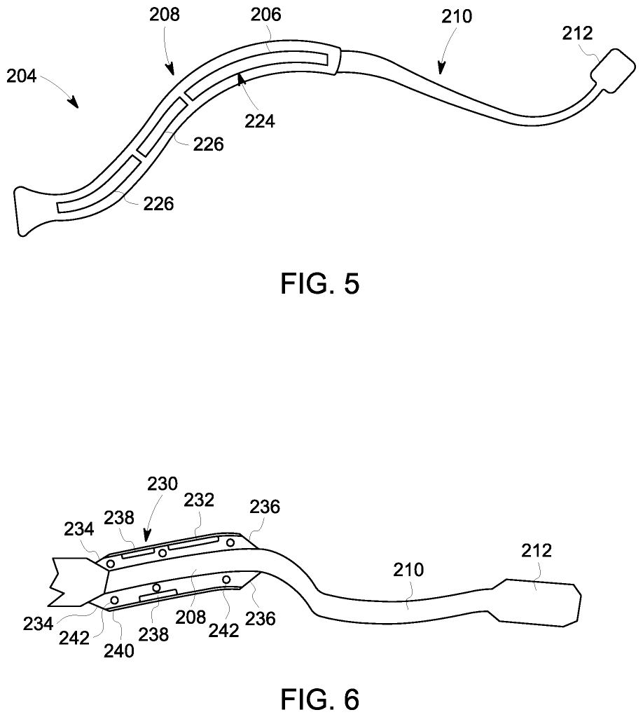

In other alternative exemplary embodiments, such as the illustrated exemplary embodiment of FIG. 5, the first section 208 can be formed with a shape memory material including shape memory alloys, such as Nitinol®, and shape memory polymer, forming at least a portion of the first section 208. For example, the first section 208 can be formed similarly to the second section 210, but can include an elongate strip 224, or multiple separate and/or attached strips and/or segments 226, of the shape memory material within the first section 208 that provide the desired flexibility and stiffness combination for the first section 208. Further, the segments 226 can be formed with different stiffness profiles or attributes, enabling different segments 226 to provide different levels of stiffness and/or flexibility to the first or proximal section 208, or portions thereof, such as with increasing flexibility present in segments 226 disposed closer to the distal section 210, for example.

Referring now to FIG. 6, in another exemplary embodiment of the disclosure, the insertion tube 204 is formed with the first or proximal section 208 and the second, or distal section 210. The first section 208 and the second section 210 are formed similarly to one another, and in an exemplary embodiment are unitarily formed with each other. A support structure 230 is disposed around the exterior of the first section 208, and can have a length similar to that of the first section 208, as discussed previously, or a length shorter or longer than the first section 208, as desired. The support structure 230 has a flexible, shape-retaining construction that has a stiffness greater than that of the first section 208 and the second section 210. The support structure 230 provides the ability to position the support structure 230 and the first section 208 disposed therein in a desired position by manipulating the support structure 230 into the desired position, and to retain the support structure 230 and the first section 208 in the desired position due to the stiffness of the support structure 230. In the illustrated exemplary embodiment of FIG. 6, the support structure 230 has a central body 232 disposed around the first section 208, with a first, proximal closure 234 at one end and a second, distal closure 236 at the opposite end. In the illustrated exemplary embodiment of FIG. 6 the first closure 234 is sized and/or adapted to engage the end of the control handle 202 adjacent the insertion tube 204, or a transitional coupling 235 securing the insertion tube 204 to the control handle 202, while the second closure 236 is sized and/or adapted to engage the insertion tube 204. The first closure 234 and the second closure 236 can be formed similarly to one another and can be formed to provide a fluid-tight seal between the closure 234,236 and the handle 202 or tube 204.

The central body 232 has a construction with a stiffness greater than that of the insertion tube 204, but that is also flexible to enable the body 232 and first portion 208 of the insertion tube 204 located within the body 232 to be manipulated into the desired configuration. In certain exemplary embodiments, the body 232 can include one or more sections 238 of a shape memory material, such as a shape memory metal or polymer, that extend along the body 232. The sections 238 can be formed with similar stiffness properties, or can have different stiffness properties. For example, the sections 238 can have increased stiffness adjacent the control handle 202 to provide more resistance to movement body 232 and first section 208 near the handle 202, and decreased stiffness near the second section 210, to provide less resistance to movement of the body 232 and first section 208 closer to the second section 210. In other alternative embodiments, the body 232 can be formed with an alternative construction that has the desired degree of stiffness while being flexible to be positioned in desired configurations, such as, but not limited to, a gooseneck construction.

The body 232 can also have various constructions with regard to the manner that the body 232 is secured to the first section 208. For example, in the illustrated exemplary embodiment shown in FIG. 6, the body 232 can be formed as a tubular member 240 that can be slid directly over the insertion tube 204. The tubular member 240 can be formed with a cross-sectional shape that conforms to the shape of the insertion tube 204/first section 208, such as a circular cross-section that conforms to the shape of the insertion tube 204/first section 208 or an oval cross-section in which the short axis conforms to the shape of the insertion tube 204/first section 208. Additionally, the interior of the tubular member 240 can include various engaging structures 242 extend between the tubular member 240 and the first section 208 and that operate to engage and hold the tubular member 240 in a stationary position on the insertion tube 204/first section 208 in addition to the engagement of the closures 234,236.

In other alternative constructions, the body 232 can be formed from a pair of opposed halves (not shown) joined to one another, such as by a hinged or separable connection, and releasably securable around the insertion tube 204/first section 208. The joined halves of the body 232 can be secured around the insertion tube 204/first section 208 as a result of the inherent properties/resiliency of the material(s) forming the body 232, or can be accomplished using a suitable closure mechanism (not shown) disposed between the opposed halves. Additionally, the body 232 can be formed as an elongate strip or wrap (not shown) of or including a suitable shape memory material that can be releasably positioned around the insertion tube 204/first section 208.

In use, when performing an interventional procedure utilizing the interventional device 106/ultrasound probe 200, after connection of the insertion tube 204 to the control handle 202, the first section and/or support structure 230 is manipulated to place the first section 208 in a desired configuration. The second section 210 can then be manipulated by the user to position the second section within the patient to obtain images of internal structures of the patient. At any point during the procedure, the first section 208 and/or support structure 230 can be re-manipulated by the user to position the first section 208 in another desired configuration to facilitate the operation of the interventional device 106/ultrasound probe 200 in the performance of the interventional procedure. After completing the procedure and removing the second section 210, the support structure 230 and/or insertion tube 204 can be detached from the interventional device 106/ultrasound probe 200, such as for sterilization for additional use or for disposal.

In another exemplary embodiment of the invention, the first section 208 and/or support structure 230 allows the insertion tube 204 to be readily employed in robotic implementations for the interventional device 106/ultrasound probe 200. As the movement mechanism (not shown) disposed within the insertion tube 204 is able to be remotely controlled to move the second section 210 when positioned within the patient 213, the same or a similar remote control mechanism (not shown) such as a robot, can be employed with the construction 217 of the first section 208 and/or support structure 230, and optionally a separate movement mechanism (not shown) associated with the first section 208 and/or support structure 230, to provide remote and/or robotic control of the position and movement of the first section 208 and/or support structure 230 in an interventional procedure.

In another exemplary embodiment of the invention, the handle 202 of the interventional device 106/ultrasound probe 200 of FIG. 2 and its control element(s) 206 are replaced by a robotic controls unit (not shown) that enable to control the various operations of the internal movement and imaging mechanisms (not shown) disposed within hollow interior of the insertion tube 204. The robot can control the sole movement of the first section 208 and/or support structure 230 or the sole movement of the second section 210 including the imaging element 111 of the tip 212 or the movement of both first 208 and second 210 sections.

The written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. An insertion tube assembly for an interventional medical device, the insertion tube assembly comprising:
a first section adapted to be secured to a handle of the interventional device and forming a proximal portion of the insertion tube; and
a second section operably connected to the first section and forming a distal portion of the insertion tube adapted for insertion into an object;
wherein the first section has a stiffness greater than the second section.

2. The insertion tube assembly of claim 1, wherein the first section has a flexible, shape-retaining construction.

3. The insertion tube assembly of claim 2, wherein the flexible shape-retaining construction is a gooseneck construction.

4. The insertion tube assembly of claim 2, wherein the flexible-shape-retaining construction includes at least one segment of a shape memory material.

5. The insertion tube assembly of claim 4, wherein the flexible, shape-retaining construction include a number of interconnected segments of the shape memory material.

6. The insertion tube assembly of claim 2, wherein the first section has a length of at least 40 cm.

7. The insertion tube assembly of claim 1, further comprising a flexible, shape-retaining support structure disposed on the first section.

8. The insertion tube assembly of claim 7, wherein the support structure comprises:
a body disposed around an exterior of the first section; and
at least one closure disposed at an end of the body, the at least one closure engaged with the first section.

9. The insertion tube of claim 8, wherein the body comprises at least one section of a shape memory material.

10. The insertion tube assembly of claim 9, wherein the body comprises a number of segments of a shape memory material.

11. The insertion tube assembly of claim 10, wherein the number of segments of shape memory material have different stiffness properties.

12. The insertion tube of claim 7, wherein the support structure is releasably disposed on the first section.

13. An interventional medical device comprising:
a control handle; and
an insertion tube assembly operably connected to the control handle, wherein the insertion tube assembly comprises:
a first section connected to the control handle and forming a proximal portion of the insertion tube; and
a second section operably connected to the first section opposite the handle and forming a distal portion of the insertion tube adapted for insertion into an object;
wherein the first section has a stiffness greater than the second section.

14. The interventional medical device of claim 13, wherein the first section has a flexible, shape-retaining construction.

15. The interventional medical device of claim 14, wherein the flexible-shape-retaining construction includes at least one segment of a shape memory material.

16. The interventional medical device of claim 13, further comprising a flexible, shape-retaining support structure disposed on the first section.

17. The interventional medical device of claim 13, wherein the interventional medical device is selected from the group consisting of an ultrasound probe, a laparoscope, a bronchoscope, a colonoscope, a needle, a catheter, an endoscope and combinations thereof.

18. The interventional medical device of claim 13, wherein the interventional medical device is a transesophageal echocardiography probe and the first section has a gooseneck construction.

19. A method for adjusting the position of an insertion tube of an interventional medical device during use in an interventional procedure for a patient, the method comprising the steps of:
providing interventional medical device comprising:
a control handle; and
an insertion tube assembly operably connected to the control handle,
wherein the insertion tube assembly comprises:
a first section; and
a second section operably connected to the first section;
wherein the first section has a stiffness greater than the second section;
manipulating the first section to position the first section in a desired configuration; and
manipulating the second section to position the second section within the patient at a desired location to obtain images of internal structures of the patient,
wherein the first section remains substantially stationary during manipulation of the second section.

20. The method of claim 19, wherein the insertion tube assembly further comprises a support structure adapted to be disposed around the first section, and wherein the method further comprises the step of placing the support structure around the first section prior to manipulating the first section.

21. The method of claim 19, further comprising step of re-manipulating the first section into another desired configuration after manipulating the second section.
